(19) United States
(12) Patent Application Publication
(10) Pub. No.: US 2008/0071365 A1
(43) Pub. Date: Mar. 20, 2008

(54) TRANSPERICARDIAL MITRAL ANNULOPLASTY SYSTEM FOR THE TREATMENT OF ISCHEMIC MITRAL REGURGITATION

(75) Inventor: Rafael Astudillo Ley, Lidingo (SE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Astudillo Medical Aktiebolag

(21) Appl. No.: 11/533,167

(22) Filed: Sep. 19, 2006

(51) Int. Cl. A61F 2/24

(52) U.S. Cl. 623/2.11; 623/2.36

(57) ABSTRACT

A transpericardial mitral annuloplasty system for the treatment of ischemic mitral regurgitation comprising an annuloplasty device comprising a body to be placed against the pericardium adjacent the mitral valve annulus opposite the anterolateral commissure of the annulus, and an attachment member being operable to tautly connect the body to the pericardium thereby causing an impression of the pericardium and the mitral valve annulus so that the annulus becomes remodeled.

### BACKGROUND OF THE INVENTION

1) Field of the Invention

The present invention refers to a transpericardial mitral annuloplasty system for the treatment of ischemic mitral regurgitation.

2) Description of Related Art

Consequences and implications of heart failure Heart failure is one of the most serious consequences of cardiovascular disease and has rapidly become one of the most important health problems in cardiovascular medicine. It affects 4.8 million people in the U.S. alone (1.5 to 2% of the U.S. adult population), and some 400,000 to 700,000 new cases are diagnosed each year.

The incidence more than doubles each decade from age 45 to 75 years, and the disease represents the most common medical discharge diagnosis for patients over age 65 years. Approximately 75% of all ambulatory patients with heart failure are 60 years of age or older. The disorder is associated with significant mortality from sudden death and progressive heart failure, with approximately 250,000 patients dying each year.

The economic impact of heart failure is significant. The large number and often high complexity of hospitalisations for heart failure make this diagnosis very costly.

Mitral regurgitation is an abnormality of various aetiologies that, if untreated, leads to myocardial dysfunction, heart failure, and sudden death. Drug treatment does not affect the prognoses of mitral regurgitation and the current accepted therapy for severe mitral regurgitation is surgical valve replacement or repair. When mitral regurgitation is not secondary to ischemic sequelae, generally accepted indications for surgery include any symptoms, left ventricular or right ventricular dysfunction or left ventricular geometric variations that reach defined levels of prognostic concern, or development of atrial fibrillation.

Ischemic mitral regurgitation remains one of the most challenging management problems in cardiac surgery with higher operative morbidity and mortality rates than for other forms of mitral valve insufficiency.

The operative approach to severe ischemic regurgitation includes open-heart surgery with revascularization (by-pass) combined with mitral valvoplasty. However, an increasing number of patients with coronary artery disease undergoing coronary angiography are treated with percutaneous coronary interventions. During this procedure a catheter introduced through the wrist or groin is used to dilate the stenotic atherosclerotic plaque in the coronary artery, and a stent is usually inserted to prevent restenosis. The introduction of drug eluting stents has significantly reduced the rate of restenosis; this has lead to a significant increase in the number of patients treated with percutaneous coronary interventions whereas the number of patients undergoing by-pass surgery is decreasing.

The shift towards less invasive treatment of coronary artery disease reveals a need of a less invasive approach for treating mitral regurgitation, where the function of the mitral valve can be restored without using an open heart surgery approach.

### BRIEF SUMMARY OF THE INVENTION

An object of the invention is to provide a new system and minimally invasive approach for the treatment of ischemic mitral regurgitation in order to restore an insufficient mitral valve.

A transpericardial mitral annuloplasty system of one embodiment the present invention is a novel method for the treatment of mitral valve annulus dilatation due to ischemic heart disease. This procedure may offer patients with coronary artery disease treated with percutaneous coronary intervention procedures and having concomitant ischemic mitral regurgitation a less invasive treatment option which avoids the risk complications associated with open-heart surgery with mitral valve repair or replacement.

The transpericardial mitral annuloplasty system according to one embodiment of the invention is characterized in that it comprises an annuloplasty device having
a body to be placed against the pericardium adjacent the mitral valve annulus opposite the anterolateral commissure of said annulus, and
an attachment member being operable to tautly connect said body to the pericardium thereby causing an impression of the pericardium and the mitral valve annulus so that said annulus becomes remodeled and the mitral valve obtains normal or substantially normal, closing condition.

### BRIEF DESCRIPTION OF THE SEVERAL VIEWS OF THE DRAWING(S)

The invention will be described further in the following with reference to the drawings, wherein:

FIG. 1 is a cross section view of the heart illustrating the mitral valve in its normal condition for closing;

FIG. 2 is an enlarged view of the mitral valve according to FIG. 1;

FIG. 3 is an end view of a device according to one embodiment of the present invention;

FIG. 4 is a perspective view of the device of FIG. 3, seen from above;

FIG. 5 illustrates the mitral valve according to FIG. 1 in a dilated, insufficient condition;

FIG. 6 illustrates the mitral valve according to FIG. 5 and provided with the device according to FIGS. 3 and 4 tautly attached to the pericardium to press onto the annulus of the mitral valve thereby restoring the mitral valve to its normal condition for closing; and

FIG. 7 is a perspective view of the heart illustrating the position of the device as attached to the pericardium in accordance with FIG. 6.

### DETAILED DESCRIPTION OF THE INVENTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Selected observations by the inventor(s) about mitral valve physiology, cardiac catheterization and echocardiography are relevant to development of embodiments of the present invention.

### Physiology

The maximal dimensions of the normal mitral valve at end-diastole are: orifice area 7.1 cm², diameter 3.0 cm and circumference 9.3 cm. A normally functioning mitral valve allows blood to flow into the left ventricle during ventricular diastole and prevents blood moving retrograde from the ventricle to the left atrium during systole. The mitral annulus is elliptical in systole and round shaped in diastole.

Mitral regurgitation occurs when the leaflets do not meet correctly allowing blood leaking backwards into the circuit of the lungs each time the heart contracts.

With chronic regurgitation, volume overload is usually tolerated very well for years before symptoms of failure develop. Left atrium enlargement predisposes patients to the onset of atrial fibrillation with the subsequent complication of embolisation. In addition, mitral valve prolapse and coronary artery disease have become major mechanisms for incompetence of the mitral valve.

Ischemia is responsible for 3%-25% of mitral regurgitation.

### Cardiac Catheterization and Echocardiography

Angiography and echo-Doppler are considered to be the criterion standard in the assessment of the severity of the mitral regurgitation.

Mitral regurgitation is graded on a scale from 0 (none); 1 (mild); 2 (moderate); 3 (moderately severe); to 4 (severe).

The severity is based on the opacity of the left atrium.

The regurgitant volume can be calculated based on information from the catheterization.

### The Device

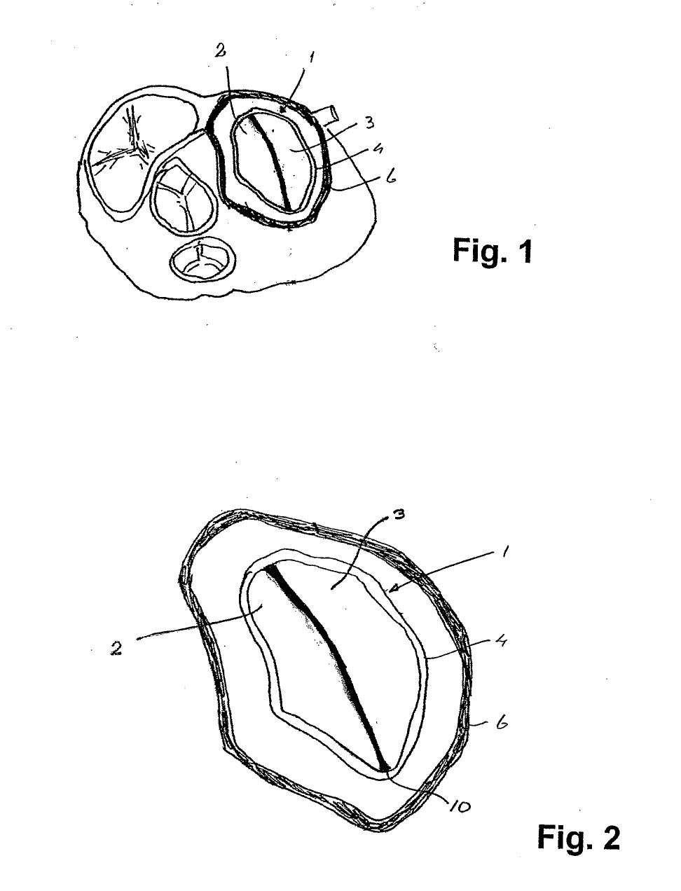

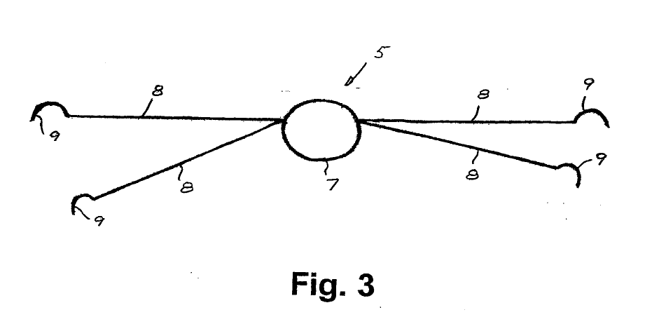

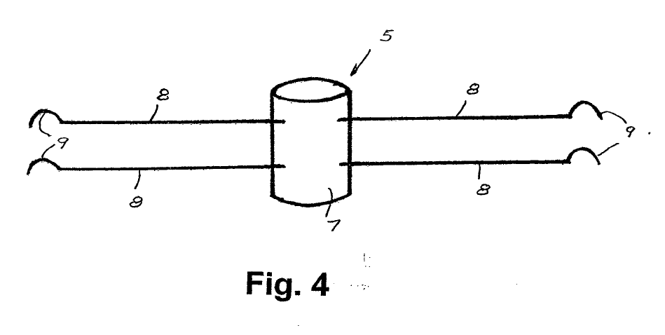

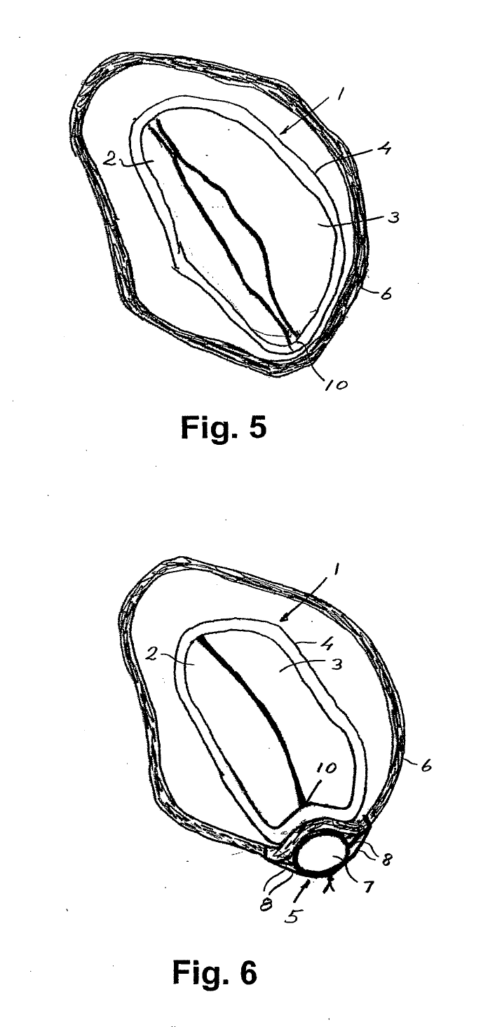

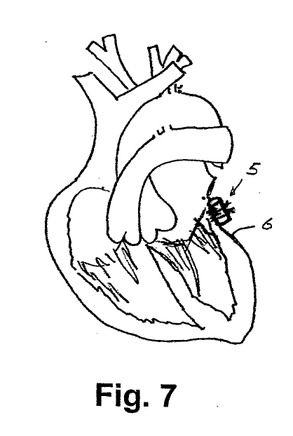

With reference to FIGS. 1 and 2, the mitral valve 1 comprises two valve leaflets 2, 3 (anterior and posterior leaflets) and a ring 4 around the mitral valve 1, known as the mitral valve annulus. The valve leaflets 2, 3 are prevented from relapsing into the left atrium by the subvalvular apparatus.

In accordance with one embodiment of the present invention the mitral valve annulus 4 are supported by a device 5 creating an impression on the outer pericardium 6 and consequently the mitral valve annulus 4 in order to give back the original and natural saddle shape of the annulus 4 which has dilated, as well as to diminish the consequently increased valve orifice area due to annulus dilation in patients with ischemic coronary disease conditions.

The device 5 is attached and sewn preferably extrapericardially, in the left side of the heart, at the anatomical place where the mitral valve annulus 4 lies closest to the pericardium 6 (anterolateral commissure), i.e. beneath the phrenic nerve and above the left pulmonary veins. Mitral regurgitation, i.e. blood flow moving backwards into the left atrium, due to a lack of insufficient closure of the leaflets 2, 3 will diminish and/or disappear with the use of the transpericardial mitral annuloplasty system.

Furthermore, implantation of the device will preferably be performed with thoracoscopically technique and through three or four small incisions on the left side of the thoracic wall. No median sternotomy neither extra-corporeal circulation support will be needed, thus avoiding all complications related to such a use.

Simultaneously, the ischemic coronary disease will be treated with percutaneous coronary intervention technique, resulting in a complete form of treatment which is minimal invasive and without the use of extra-corporeal circulation. This sub-group of patients with ischemic coronary disease and concomitant mitral regurgitation on basis of a dilated mitral valve annulus 4 are according to present guidelines treated with open-heart surgery. The use of the transpericardial mitral annuloplasty system can offer a new minimally invasive therapeutic form of treatment for patients with coronary artery disease and mitral regurgitation caused by a dilated mitral valve annulus 4.

The device comprises a body 7 which has a suitable cross section, such as an elliptical or oval form with a length of about 2.5 cm and a width (largest cross dimension) of about 1.75 cm, for instance. The body 7 is provided with an attachment member in form of one or several (e.g., 3, 4 or 5) different surgical threads 8 and respectively needles 9. The threads 8 are not coming out exactly at the midline seen from the sidewall of the device 5 but somewhat backwards closer to the pericardial wall. Other types of attachment members may be used.

In other embodiments, the body 7 is a cylinder of approximately 1.75 to 2.0 cm in diameter and 2.0 to 3.0 cm long. This size may be varied based on the size of the heart and its inner structures displacement.

The body 7 of the device 5 may be made of a silicone material, a plastic material or a metal material. Preferably the silicone is a hard silicone material. The body 7 may be made inflatable and/or shape-changeable in such a way that the body 7 can be remodeled afterwards in situ if desired. The body 7 may be inflated, for instance, by way of a fluid, such as an appropriate liquid solution that is introduced into the body 7 through a long needle across the thoracic wall while controlling the process with tomography computerized guided technique and without the need of a surgical operation.

The correct positioning of the device 5 is decided after testing with a light pressure at different places in the aforementioned area on the left side of the pericardium 6.

The effect on valve regurgitation will be controlled simultaneously with the transesophageal echocardiography Doppler investigation. From the decided position, the needles 9 will pass through the pericardium 6 at a point between 1.5 to 2.0 cm from the outer edge of the body 7; the sutures 8 will then be tied in a surgical knot fashion at the outer surface of the body 7 such that the threads 8 will exert forces onto the body 7, said forces being transferred by the body 7 to the annulus 4 via the pericardium 6 as is illustrated in FIG. 6. Transesophageal echocardiography is used to control the tightening of the surgical threads and tension on the knots until valve regurgitation disappears.

With the consistently effect of pleating the pericardium around the device, the elastic tension of the pericardium 6 with the body 7 on place will then cause an impression protruding medially into the heart, this same impression will press the adjacent mitral valve annulus 4 close to its anterolateral commissure 10.

This mechanical effect on the annulus 4 will be twofold, firstly the real orifice area of the mitral valve 1 will decrease, secondly the annulus 4 will gain again its original saddle shape with the consequent disappearance of the regurgitation.

The transpericardial mitral annuloplasty system benefits from an assembly or holder for thoracoscopically holding the body 7 of the annuloplasty device 5 in a substantially taut position for the extrapericardial pleating with the help of the surgical threads 8 and needles 9 at an aforementioned place close to the anterolateral mitral valve commissure 10.

The assembly includes a portion, which will hold the body 7 of the device 5 and is positioned and held in a 90 degrees shape against and substantially equivalent to at least a portion of the valve annulus 4.

The assembly further includes a mechanism for releasably binding the body 7 of the annuloplasty device 5 to this surface and be held in a taut position against the pericardium 6.

What is also helpful is an assembly herein denoted pericardial-lifting instrument for thoracoscopically use, which is round (circumferential) ended, including three or more small point needles like surface orientated in such a way that with a light clockwise rotation of this instrument against the pericardium 6, its surface will be caught and lifted, making it easier to pass the pericardium 6 with the needles 9 without the risk of injuring the underlying surface of the heart.

After positioning the needles 9, their respective threads 8 and the body 7 itself, the instrument will be rotated counterclockwise and consequently releasing the substantially tautly held pericardium 6.

Also included may be an optical device for viewing placement of the device 20. Correct positioning of the device is decided after testing with a light pressure at different places in the area on the left side of the pericardium that triggers a maximal point of effectiveness.

Positioning outside the mediastinum (heart and big vessels) has the advantages of being less invasive, reducing infection rates, reduced cardiac rhythm disturbances and an easier technical implementation.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A transpericardial mitral annuloplasty system comprising:
an annuloplasty device for the treatment of ischemic mitral valve regurgitation, said annuloplasty device including:
a body configured for placement against a pericardium adjacent an annulus of the mitral valve, and
at least one attachment member operable to tautly connect said body to the pericardium thereby causing an impression of the pericardium and the mitral valve annulus so that said mitral valve annulus becomes remodeled and the mitral valve obtains normal or substantially normal, closing condition.

2. A system according to claim 1, wherein the body is further configured for placement opposite an anterolateral commissure of said annulus

3. A system according to claim 1, further comprising a holder having a groove formed with a grip surface shaped to hold said annuloplasty device and a releasable retainer for tautly holding the annuloplasty device against said groove at the pericardial surface.

4. A system according to claim 3, further comprising a pericardium lifting instrument with a releasable retainer, said releasable retainer being operable to selectively grip and release the pericardial surface from said pericardial holder.

5. A system according to claim 1, wherein said attachment member comprises a plurality of threads for attachment to the body and needles connected to outer ends of the threads.

6. A system according to claim 1, wherein the body has a cylindrical shape.

7. A system according to claim 6, wherein the cylindrical shape has an elliptical cross-section.

8. A holder for use with an annuloplasty device, said holder comprising:
a body defining a groove formed with a grip surface shaped to hold the annuloplasty device.

9. A holder according to claim 8 further comprising a releasable retainer for tautly holding the annuloplasty device within said groove.

10. A holder of claim 9, wherein the groove has a cylindrical shape.

11. A method of treating ischemic mitral valve regurgitation comprising:
inserting an annuloplasty device comprising a body and an attachment member into the heart;
placing said body against a pericardium adjacent a mitral valve annulus and opposite an anterolateral commissure of said annulus; and
connecting said body tautly to the pericardium using the attachment member and causing an impression of the pericardium and the mitral valve annulus so that said annulus becomes remodeled and the mitral valve obtains normal or substantially normal, closing condition.

12. A method of claim 1 1, wherein placing said body includes placing the body thoracoscopically and without the help of heart lung machine.

13. A method of claim 11, further comprising gripping said body within a groove of a holder before inserting the annuloplasty device.

14. A method of claim 13, wherein gripping said body includes tautly holding the annuloplasty device within the groove of the holder using a retainer

15. A method of claim 14, further comprising releasing the body from the groove by releasing the retainer after connecting the body.

16. A method of claim 1 1, further comprising lifting the pericardium using a retainer of a lifting instrument before placing the body against the pericardium.

17. A method of claim 16, further comprising releasing the retainer of the lifting instrument after connecting the body.

18. A method of claim 17, wherein the lifting instrument is used thoracoscopically.
